(19) United States
(12) Patent Application Publication
(10) Pub. No.: US 2007/0004037 A1
(43) Pub. Date: Jan. 4, 2007

(54) Methods and compositions for culturing keratinocytes

(76) Inventor: Rodolfo Faudoa, San Antonio, TX (US)

Correspondence Address: WILSON SONSINI GOODRICH & ROSATI, 650 PAGE MILL ROAD, PALO ALTO, CA, 94304-1050, US

(21) Appl. No.: 11/258,346

(22) Filed: Oct. 24, 2005

Related U.S. Application Data
(60) Provisional application No. 60/696,141, filed on Jul. 1, 2005.

(51) Int. Cl. C12N 5/08; C12N 5/06

(52) U.S. Cl. 435/325; 435/366

(57) ABSTRACT

Methods, compositions, and kits are disclosed for culturing keratinocytes and for the treatment of wounds and burns, as well as cosmetic compositions. The methods and compositions include the use of conditioned medium from the culture of keratinocytes, where the medium may be used to further culture keratinocytes or used in compositions such as keratinocyte culture medium and pharmaceutical and cosmetic compositions.

### CROSS REFERENCE

This application claims the benefit of U.S. Provisional Application No. 60/696,141 filed Jul. 1, 2005, which is incorporated herein by reference in its entirety.

### STATEMENT AS TO FEDERALLY SPONSORED RESEARCH

The inventor was supported in part with government support under Grant No. RO1GM041343 awarded by the National Institutes of Health. The United States may have certain rights in this invention.

### BACKGROUND OF THE INVENTION

Two decades ago, the emergence of the biotechnology industry sparked the development of methods for large-scale cell culture, as companies raced to produce therapeutic quantities of the first recombinant proteins. Continuous refinements to cell culture techniques, instrumentation, and quality control measures ultimately have made large-scale cell culture more a science than an art. Cell culture is vital no only to the development of new pharmaceuticals, but to the testing of existing therapeutics, the advancement of tissue engineering and transplant, and the rigorous understanding of biologic and physiologic systems, including cancer.

There are a number of growth limitations for normal cells grown in culture. To circumvent these limitations, researchers and biotechnology manufacturers often employ ‘immortalized’ (i.e. genetically transformed/mutated) cell lines for the development, testing and manufacturing of therapeutics that will eventually be used to target ‘normal’ human cells. There are many benefits of cell lines such as cost, reproducibility, and longevity in culture. The one main problem is that cell lines are not normal, but simply attempt to simulate normal cell function. This may account for some of the side effects encountered by humans when taking therapeutics developed and tested using cell lines.

Normal cells do not grow ‘ideally’ in traditional culture media. Therefore, technology changed the cell to fit the growth medium. There is still a need to change the medium to fit the cell. For many applications, e.g., the use of keratinocyte culture for research and/or therapeutic purposes, cell lines are not adequate, and current methods for culture often require use of serum, or produce cell cultures that are mixtures of keratinocytes and fibroblasts. Cultures of human keratinocytes are increasingly being used in examinations of skin structure and disease, and as in vitro models of human skin in toxicology studies (Boyce, S. T., and Ham, R. G., in: In Vitro Models for Cancer Research, vol. III, Webber, M. M., et al., eds., Boca Raton, Fla.: CRC Press, Inc., pp. 245-274 (1985)). Successful culture of keratinocytes has proven, however, to be somewhat difficult, owing primarily to their nutritional fastidiousness (Gilchrest, B. A., et al., J. Cell. Physiol. 120:377-383 (1984)). For example, in most early studies using traditional serum-supplemented culture media, keratinocytes from skin explants were rapidly overgrown by less fastidious and faster-growing fibroblasts that were also resident in the tissue (Freshney, Id.). Thus, there has been substantial work expended in the attempt to formulate culture media favoring the selection and successful in vitro cultivation of human keratinocytes. This is especially true in the culture of fetal keratinocytes, which have potential usefulness in, e.g., wound healing.

### SUMMARY OF THE INVENTION

The invention provides methods, compositions, kits, and business methods for culturing cells by contacting the cells with conditioned medium, and for treatment of wounds.

In one aspect, the invention provides methods. In some embodiments, the invention provides a method of culturing keratinocytes that includes contacting growing keratinocytes with a culture medium containing conditioned medium, where the conditioned medium has previously been used to culture fetal keratinocytes. In some embodiments, the method further includes contacting the growing keratinocytes with a cyclic adenosine monophosphate (cAMP)-elevating agent, e.g., cAMP-elevating agent selected from the group consisting of forskolin, cholera toxin, dibutyryl cAMP, isobutylmethylxanthine, theophylline, isoproterenol, and PGE2. In some embodiments, the cAMP-elevating agent is forskolin. In some embodiments, the forskolin is present at a concentration of about 0.8 ug/ml to about 5 ug/ml. In some embodiments, the growing keratinocytes are fetal keratinocytes. In some embodiments, the growing fetal keratinocytes and the fetal keratinocytes used to produce the conditioned medium are genetically identical. In some embodiments, the culture medium comprises about 30-50% conditioned medium. In some embodiments, the culture medium is produced by the steps of: (a) culturing fetal keratinocytes in a keratinocyte culture medium for at least about eight hours to produce a conditioned medium; and (b) mixing the conditioned medium with fresh keratinocyte culture medium so that the conditioned medium comprises about 30-50% by volume of the mixture. In some of these embodiments, adding a cAMP-elevating agent to the culture medium is further included. In some embodiments, step (b) is repeated about every day. In some embodiments, step (b) is repeated until a cell culture is produced wherein the cells of the cell culture comprise greater than about 99% keratinocytes. In some embodiments, step (b) is repeated until a cell culture is produced wherein the cells of the cell culture comprise greater than about 99.9% keratinocytes. In some embodiments the methods further include passing the keratinocytes when they are about 60-70% confluent. In some embodiments, the fetal keratinocytes are grown in serum-free medium.

In some embodiments, the invention provides a method of culturing keratinocytes comprising contacting growing keratinocytes with a culture medium comprising conditioned medium, where the conditioned medium has previously been used to culture keratinocytes under animal product-free conditions at all times. In some embodiments the method further includes contacting the growing keratinocytes with a cAMP-elevating agent.

In another aspect, the invention provides compositions. In some embodiments, the invention provides a composition for culturing keratinocytes that includes keratinocyte culture medium and conditioned medium in which fetal keratinocytes have been cultured In some embodiments, the composition further includes a cAMP-elevating agent, e.g., an agent selected from the group consisting of forskolin, cholera toxin, dibutyryl cAMP, isobutylmethylxanthine, theophylline, isoproterenol, and PGE2. In some embodiments, the cAMP-elevating agent is forskolin. In some embodiments, the forskolin is present at a concentration of about 0.8 ug/ml to about 5 ug/ml. In some embodiments, the conditioned medium comprises about 30-50% of the composition by volume.

In some embodiments, the invention provides a culture medium produced by mixing fresh culture medium with a protein produced by the process of culturing fetal keratinocytes. In some embodiments, the composition further includes a cAMP-elevating agent.

In some embodiments, the invention provides a cell culture medium that contains conditioned medium that has previously been used to culture keratinocytes under conditions where the cultured keratinocytes are cultured at all times under animal product-free conditions. In some of these embodiments the medium further includes a cAMP-elevating agent.

In some embodiments, the invention provides a keratinocyte culture that persists for more than about one year, wherein the cells of the culture comprise at least about 99% keratinocytes.

In some embodiments, the invention provides a composition for promoting wound healing that contains a protein that is produced by growing fetal keratinocytes in culture.

In some embodiments, the invention provides a composition for promoting wound healing that contains a protein that is produced by growing keratinocytes in culture, where the keratinocytes are grown by a method that includes contacting growing keratinocytes with a culture medium containing conditioned medium, where the conditioned medium has previously been used to culture keratinocytes under conditions where the cultured keratinocytes are cultured at all times under animal product-free conditions.

In some embodiments, the invention provides a method of modulating wound healing by contacting a wound with a protein from a conditioned medium produced by fetal keratinocyte culture. In some embodiments, the method further includes contacting the wound with a cAMP-elevating agent. In some embodiments, the cAMP-elevating agent is forskolin.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

FIG. 1 is a graph depicting proliferation of adult keratinocytes and adult fibroblasts in culture over time.

FIG. 2 is a graph depicting proliferation of fetal keratinocytes and fibroblasts in culture over time, with and without conditioned medium.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

### DETAILED DESCRIPTION OF THE INVENTION

I. Introduction

The invention encompasses methods and compositions that utilize keratinocyte-conditioned medium in the culture of keratinocytes and for therapeutic and other purposes. The methods and compositions can be used, e.g., for the development and testing of novel pharmacologic therapeutics and commercial production of cell therapeutics. In some embodiments the methods and compositions also utilize an exogenously-added cAMP elevating agent. The keratinocytes being cultured, or used to produce conditioned medium, may be of adult, neonatal, or fetal origin, and may be from humans or animals. In some embodiments the keratinocytes are of fetal origin. The methods of the invention include methods allowing long-term culture of highly purified keratinocyte cultures, especially fetal keratinocyte cultures. The compositions of the invention include culture media that contain keratinocyte-conditioned medium, and therapeutic and cosmetic compositions containing keratinocyte-conditioned-medium or factors derived from keratinocyte-conditioned medium. The medium may be used as is, mixed with other components, in concentrated or lyophilized form, or in partially or completely purified form The present invention also provides kits, e.g., kits for culturing keratinocytes. The invention further provides business methods based on the methods and compositions utilizing conditioned medium from keratinocyte culture. In some embodiments, the methods and compositions utilize serum-free and/or animal product-free culture media.

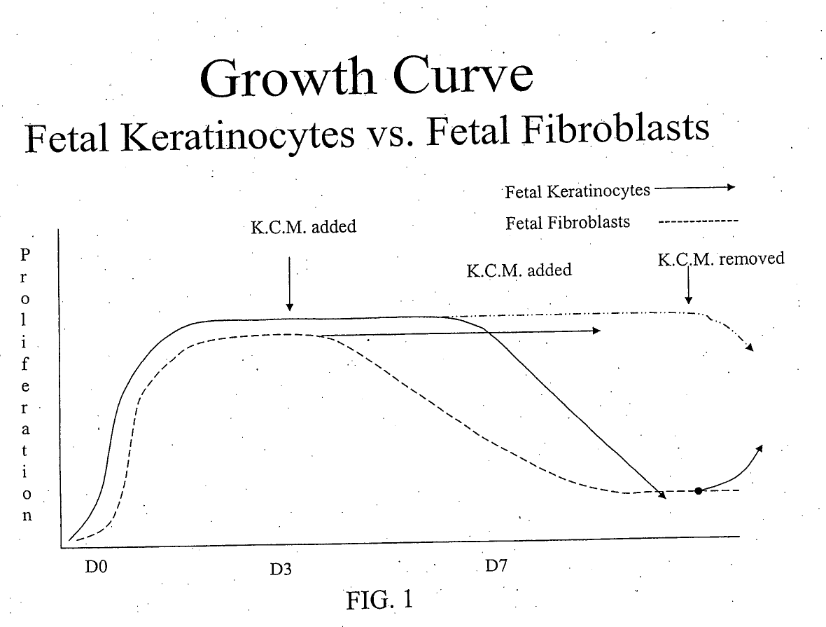

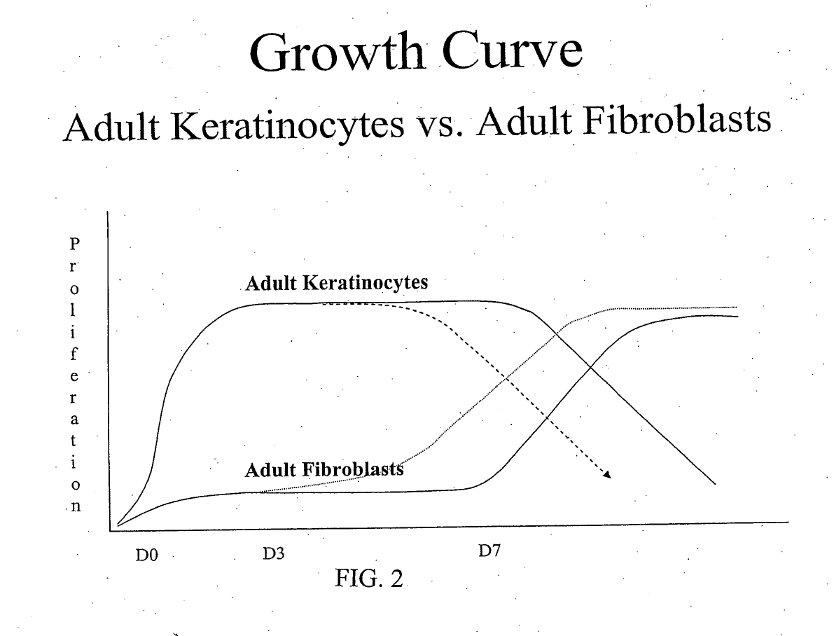

FIGS. 1 and 2 illustrate the effects of compositions and methods of the invention on the culture of keratinocytes. FIG. 1 illustrates the typical result of culturing keratinocytes over time; the culture is overrun by fibroblasts, which are more robust and eventually displace the keratinocytes. FIG. 2 illustrates the same results for fetal keratinocytes, which are much more difficult to separate from fibroblasts for seeding in primary culture. However, in FIG. 2 the effects of adding keratinocyte-conditioned medium (KCM) and a cyclic AMP-elevating factor (forskolin) to the fetal keratinocyte culture are also illustrated. The KCM and forskolin act to decrease the concentration of fibroblasts in the culture (by inducing apoptosis of fibroblasts) without adversely affecting the keratinocytes. If KCM is removed, fibroblasts again begin to proliferate, displacing keratinocytes. The methods and compositions of the invention are based on the ability of KCM and a cAMP-elevating agent to decrease fibroblast contamination while allowing robust growth of keratinocytes.

II. Keratinocyte Sources

The methods and compositions of the invention utilize keratinocytes or keratinocyte-derived substances. As used herein, “keratinocytes” encompasses the specialized epithelial cells found in the epidermis of the skin that synthesize keratin. Unless otherwise indicated, keratinocyte cell lines are not included within this definition. In the upper, cornified layers of the skin (those exposed to the environment), the cytoplasm of the keratinocytes is completely replaced with keratin and the cells are dead. The keratinocytes located in the lower layers, however, particularly in the basal epidermis (stratum basale), actively divide and ultimately migrate up through the more superficial layers to replace those cells being sloughed off at the external surface. Accordingly, the skin can be thought of as a dynamic organ comprising keratinocytes that are constantly dividing, maturing and ultimately dying.

Any source of keratinocytes may be used in the methods and compositions of the invention. The keratinocytes may be of animal or human origin, and may be from fetal, newborn, juvenile, or adult organisms. Typically, the initial source of keratinocytes is skin. Skin can be obtained by appropriate biopsy or upon autopsy. In the case of animal skin, the animal may be sacrificed and skin removed and treated after sacrifice.

In some embodiments, the tissue (skin) is cleaned, removed and placed in appropriate medium, e.g., Dulbecco's Modified Eagle's Medium (DMEM). Other media may be used, as will be apparent to those of skill in the art. If the skin is fetal, in some embodiments the source of the skin may be mouse or rat, but any suitable source of fetal skin may be used. A common source of non-fetal, e.g., neonatal skin, is from newborn humans (e.g., foreskin removed on circumcision of males).

Skin from newborn or adult animals may be treated mechanically to remove epidermis from dermis by techniques well-known in the art, generally followed by treatment with a protease. Skin from fetal animals generally cannot be separated into dermis and epidermis by mechanical means, and can be treated by digestive enzyme(s), e.g., protease instead. Thus, either mechanically separated epidermis, or whole skin (e.g., fetal), may be treated with protease. For example, the skin or epidermis can be disaggregated mechanically and/or treated with digestive enzymes and/or chelating agents that weaken the connections between neighboring cells making it possible to disperse the tissue into a suspension of individual cells without appreciable cell breakage. Enzymatic dissociation can be accomplished by mechanically disrupting the tissue and treating the disrupted tissue with any of a number of digestive enzymes either alone or in combination. These include but are not limited to trypsin, chymotrypsin, collagenase, elastase, and/or hyaluronidase, DNase, pronase, dispase, and the like. Mechanical disruption can be accomplished by a number of methods including, but not limited to, the use of grinders, blenders, sieves, homogenizers, pressure cells, or insonators to name but a few. For a review of tissue disaggregation techniques, see Freshney, Culture of Animal Cells: A Manual of Basic Technique, 4th edition., A. R. Liss, Inc., New York, 2000, which is incorporated by reference herein in its entirety.

In one embodiment, the skin is chopped with scissors, then incubated in 0.3% Dispase & trypsin 0.2% (1/1 dilution). Incubation may be at any temperature where the protease is active; e.g., between about 5° C. and about 60° C., or about 10° C. and about 50° C., or about 20° C. and about 45° C., or about 25° C. and about 40° C., or about 30° C. and about 40° C. In some embodiments, the skin and protease are incubated at about 32, 33, 34, 35, 36, 37, 38, 39, or 40° C. In some embodiments, a temperature of about 37° C. is used. The skin/protease mixture is incubated for a length of time that allows separation of cells without significant damage to the desired cells, i.e., keratinocytes. The incubation time may be, e.g., between about 0.25 hour and about 20 hours, or about 0.5 hour and about 15 hours, or about 0.5 hour and about 10 hours, or about 0.5 hours and about 5 hours. In some embodiments, the incubation time is about 1 to about 3 hours, or about 1 to about 2 hours. In some embodiments, the incubation time is less than about 5, 4, 3, 2 or 1 hours. In some embodiments, the incubations time is about 1 hour. In some embodiments, the incubation time is about 1.5 hours. In some embodiments, the incubation time is about 2 hours. The incubation medium may be mixed, e.g., every 15-20 minutes. Surprisingly, it has been found that incubation times of about 1 to about 2 hours produce the best results for fetal skin, in contrast to longer incubation times (typically overnight) that are used in common protocols for obtaining keratinocytes.

After incubation the cells are dissociated, e.g., with a 10 ml glass pipette. The dissociated cells are passed through a cell strainer, e.g., a 75 μm strainer. The cells are washed in medium (e.g., DMEM) and centrifuged. Exemplary washing and centrifugation protocols include centrifugation at about 800 rpm (e.g., about 100×G) for 5-10 minutes, with the wash and centrifugation steps repeated at least three times.

At this point the cells are ready for culture. Optionally, the cells may be further treated to enrich the starting culture in keratinocytes by methods well-known in the art; e.g., using standard techniques for cell separation including, but not limited to, cloning and selection of specific cell types, selective destruction of unwanted cells (negative selection), separation based upon differential cell agglutinability in the mixed population, freeze-thaw procedures, differential adherence properties of the cells in the mixed population, filtration, conventional and zonal centrifugation, centrifugal elutriation (counterstreaming centrifugation), unit gravity separation, countercurrent distribution, electrophoresis and fluorescence-activated cell sorting. For a review of clonal selection and cell separation techniques, see Freshney, ibid. However, a major advantage of the methods of the present invention is that cultures become enriched in keratinocytes without the necessity for such pretreatment of the cell population to be cultured; thus, such pretreatment is optional.

Cells may be cultured immediately after preparation, or may be stored. For storage, standard protocols of the art may be used.

III. Culture Methods

Cells obtained as described are then cultured. The cells may be cultured in any manner known in the art including in monolayer, beads or in three-dimensions and in any suitable container (e.g., culture dish, roller bottle, a continuous flow system, etc.). Methods of cell and tissue culturing are well known in the art, and are described, for example, in Freshney, ibid, as well as Doyle and Griffiths, eds., Cell & Tissue Culture: Laboratory Procedures, John Wiley & Sons Ltd., Chichester, England 1998; which is incorporated herein by reference in its entirety.

The cell culture medium used for initial culture of growing keratinocytes, and/or with which conditioned medium is mixed in later stages of cell culture, may be any cell culture medium which adequately addresses the nutritional needs of the keratinocytes. In some embodiments of the invention, the culture medium is serum-free. In some embodiments of the invention, the culture medium is animal-product free. “Animal-product free” media, as used herein, includes media that do not contain products of animal origin, with the exception of products produced by the keratinocytes themselves. Examples of animal products include bovine serum albumin, bovine hypothalamic factor, and bovine pituitary factor. In some embodiments of the invention, the culture medium is a defined medium. Examples of keratinocyte culture media include, but are not limited to MCDB 153, or GIBCO Keratinocyte Serum-Free Medium, or other media formulations readily apparent to those skilled in the art, including those described in U.S. Pat. No. 6,692,961, and in Methods For Preparation of Media, Supplements and Substrate For Serum-Free Animal Cell Culture Alan R. Liss, New York (1984) and Doyle and Griffiths, ibid, all of which are incorporated by reference herein in their entirety. The medium may be supplemented with any components necessary to support the desired cell or tissue culture.

The methods of the invention allow culture of keratinocytes without the addition of serum. In some embodiments, however, it may be desirable to use serum, and the invention does not preclude the use of serum if appropriate. Thus, serum, such as bovine serum, which is a complex solution of albumins, globulins, growth promoters and growth inhibitors may be added if appropriate. The serum should be pathogen free and carefully screened for mycoplasma, bacterial, fungal, and viral contamination. Also, the serum should generally be obtained from the United States and not obtained from countries where indigenous livestock carry transmittable agents. Hormone addition into the medium may or may not be desired.

Other ingredients, such as vitamins, growth and attachment factors, proteins etc., can be selected by those of skill in the art in accordance with his or her particular need.

Other ingredients include, without limitation, amino-acids (both D and/or L-amino acids) such as glutamine, alanine, arginine, asparagine, cystine, glutamic acid, glycine, histidine, isoleucine, leucine, lysine, methionine, phenylalanine, proline, serine, threonine, tryptophan, tyrosine, and valine and their derivatives; acid soluble subgroups such as thiamine, ascorbic acid, ferric compounds, ferrous compounds, purines, glutathione and monobasic sodium phosphates. Additional ingredients include sugars, deoxyribose, ribose, nucleosides, water soluble vitamins, riboflavin, salts, trace metals, lipids, acetate salts, phosphate salts, HEPES, phenol red, pyruvate salts and buffers. Other ingredients often used in media formulations include fat soluble vitamins (including A, D, E and K) steroids and their derivatives, cholesterol, fatty acids and lipids Tween 80, 2-mercaptoethanol pyramidines as well as a variety of supplements including serum as discussed above (fetal, horse, calf, etc.), proteins (Insulin, transferrin, growth factors, hormones, etc.) antibiotics (gentamicin, penicillin, streptomycin, amphotericin B, etc.) whole egg ultra filtrate, and attachment factors (fibronectins, vitronectins, collagens, laminins, tenascins, etc.). The concentration of the ingredients are well known to one of ordinary skill in the art. See, for example, Methods For Preparation Of Media, Supplements and Substrate for Serum-free Animal Cell Cultures, supra.

In some embodiments, cells are plated in GIBCO Keratinocyte Serum-Free Medium. The medium may be used with or without bovine pituitary extract (BPE). Although culture is improved with BPE, BPE adds unknown factors from a bovine source. It is not necessary for keratinocyte culture according to the methods of the invention and its use is optional; in some embodiments, keratinocytes are cultured in media free of all animal products, including BPE. If BPE is used, it may be used at any concentration appropriate to the culture of keratinocytes. Exemplary concentrations are about 1 to about 100 ug/ml, or about 5 to about 75 ug/ml, or about 10 to about 50 ug/ml, or about 10, 15, 20, 25, 30, 35, 40, 45, or 50 ug/ml. In some embodiments, BPE is used at 10-15 ug/ml. In some embodiments, BPE is used at 30 ug/ml.

The medium is replaced at intervals. The intervals may be regular or irregular. Replacement intervals can be from about 0.25 to about 10 days, or about 0.5 to about 5 days, or about 0.5 to about 4, 3, 2, or 1 days, or about 1, 2, 3, 4, 5, 6, 7, 8, 9, or 10 days. In some embodiments, the medium is replaced about daily

In one or all of the media replacements, the media is replaced with fresh medium mixed with conditioned medium. The conditioned medium may be from the keratinocyte culture itself (e.g., medium that would otherwise be discarded) or may be from another keratinocyte culture, or may be a combination of media from two or more cultures. In some embodiments, the conditioned medium is from fetal keratinocytes. Thus, in some embodiments, the invention provides methods of culturing keratinocytes comprising contacting growing keratinocytes with a culture medium comprising conditioned medium, where the conditioned medium has previously been used to culture fetal keratinocytes. Such methods are particularly useful in the culture of fetal keratinocytes, which, as mentioned, may themselves be the source of the conditioned medium. In some embodiments, the conditioned medium is from keratinocytes, e.g., adult, neonatal, or fetal keratinocytes, that have been cultured entirely in serum-free medium. In some embodiments, the conditioned medium is from keratinocytes, e.g., adult, neonatal, or fetal keratinocytes, that have been cultured entirely in animal product-free medium. Thus, in some embodiments, the invention provides a method of culturing keratinocytes comprising contacting growing keratinocytes with a culture medium comprising conditioned medium, where the conditioned medium has previously been used to culture keratinocytes under animal product-free conditions at all times. Without being bound by theory, it is thought that the conditioned medium provides one or more factor(s) (e.g., protein) that inhibits fibroblast growth (e.g., that induce fibroblast apoptosis). This factor(s) is thought to be coming from the keratinocytes themselves. Thus, when keratinocytes are still at low confluence, there is generally a need for more conditioned medium to provide the requisite amount of the factor(s). Thus, the proportion of conditioned medium to fresh medium depends on the degree of confluence of the cells from which the medium was taken, as well as the degree of confluence of the cells to which the medium will be added. This proportion may be readily determined by the practitioner for the particular system being used and stage of culture. In some embodiments, the proportion of conditioned medium to fresh medium is about 10% to about 90% (vol %) or about 15% to about 75%, or about 20% to about 60%, or about 30% to about 60%, or about 30% to about 50%, or about 35% to about 50%, or about 35% to about 45%, or about 40% to about 50%, or about 45% to about 55%, or about 25, 30, 35, 40, 45, 46, 47, 48, 49, 50, 51, 52, 53, 54, 55, or 60%. In some embodiments, the proportion of conditioned medium to fresh medium is about 50%. In some embodiments, the proportion of conditioned medium to fresh medium may be different on different days of culture. In some embodiments, the proportion of conditioned medium to fresh medium may be the same on different days of culture. Any combination of the same or different proportions on different days may also be used. In some embodiments, the proportion of conditioned medium to fresh medium is about 50% for all media changes. The media may be replaced with media that is part conditioned media and part fresh media for about 1, 2, 3, 4, 5, 6, 7, 8, 9, 10, 11, 12, 13, 14, 15, 16, 17, 18, 19, 20, 25, 30, or more than about 30 days. In some embodiments, the media is replaced for about 4, about 5, or about 6 days. In some embodiments, the media is replaced for about 5 days.

Keratinocyte-conditioned medium may be used in making culture medium for keratinocytes, and may also be used in making compositions for therapeutic or cosmetic use. It may be desirable to gather medium when cells are at a particular stage of confluence. The particular stage of confluence can vary, depending on the intended use of the conditioned medium, as the makeup of the conditioned medium changes with degree of confluence, both in terms of the profile of proteins and other factors in the medium, and in terms of the concentrations of various proteins and other factors in the medium. Thus, in some embodiments, conditioned medium is gathered from cells that are at least about 20%, 30%, 40%, 45%, 50%, 55%, 60%, 65%, 70%, 75%, 80%, 85%, 90%, 95%, 98%, 99%, or 100% confluent. In some embodiments, conditioned medium is gathered from cells that are between about 10% and 90% confluent, or between about 20% and 80% confluent, or between about 30% and 70% confluent, or between about 40% and 60% confluent. The medium gathered at higher stages of confluence (e.g., more than about 60%) is generally no longer optimal for use in keratinocyte culture (see below), but may have use in therapeutic and other areas.

In some embodiments, the invention encompasses adding a cAMP-elevating agent to the culture, in addition to adding conditioned medium. The cAMP-elevating agent may be added at any suitable time. Typically, the cAMP-elevating agent is added at the start of culture, e.g., at day 0. As used herein, a “cAMP-elevating agent” encompasses agents that elevate intracellular cAMP levels and/or that augment or potentiate the action of cAMP. See, e.g., U.S. Pat. No. 6,610,535, the disclosure of which is incorporated by reference in its entirety. A cAMP-elevating agent that is added to the culture or culture medium, as opposed to being produced by the cells in culture, is referred to herein as “exogenous” or “exogenously-added” cAMP-elevating agent.

Useful in the invention are compounds that may activate adenylate cyclase including, but not limited to: forskolin (FK), cholera toxin (CT), pertussis toxin (PT), prostaglandins (e.g., PGE-1 and PGE-2), colforsin and β-adrenergic receptor agonists. β-Adrenergic receptor agonists include albuterol, bambuterol, bitolterol, carbuterol, clenbuterol, clorprenaline, denopamine, dioxethedrine, dopexamine, ephedrine, epinephrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, prenalterol, procaterol, protokylol, reproterol, rimiterol, ritodrine, soterenol, salmeterol, terbutaline, tretoquinol, tulobuterol, and xamoterol.

Also useful in the invention are compounds which may inhibit cAMP phosphodiesterase(s), and thereby increase the half-life of cAMP. Such compounds include amrinone, milrinone, xanthine, methylxanthine, anagrelide, cilostamide, medorinone, indolidan, rolipram, 3-isobutyl-1-methylxanthine (IBMX), chelerythrine, cilostazol, glucocorticoids, griseolic acid, etazolate, caffeine, indomethacin, theophylline, papverine, methyl isobutylxanthine (MIX), and fenoxamine.

Certain analogs of cAMP, e.g., which are agonists of cAMP, can also be used. Exemplary cAMP analogs which may be useful in the present method include dibutyryl-cAMP (db-cAMP), (8-(4)-chlorophenylthio)-cAMP (cpt-cAMP), 8-[(4-bromo-2,3-dioxobutyl)thio]-cAMP, 2-[(4-bromo-2,3-dioxobutyl)thio]-cAMP, 8-bromo-cAMP, dioctanoyl-cAMP, Sp-adenosine 3′:5′-cyclic phosphorothioate, 8-piperidino-cAMP, N⁶-phenyl-cAMP, 8-methylamino-cAMP, 8-(6-aminohexyl)amino-cAMP, 2′-deoxy-cAMP, N⁶, 2′-O-dibutryl-cAMP, N⁶, 2′-O-disuccinyl-cAMP, N⁶-monobutyryl-cAMP, 2′-O-monobutyryl-cAMP, 2′-O-monobutryl-8-bromo-cAMP, N⁶-monobutryl-2′-deoxy-cAMP, and 2′-O-monosuccinyl-cAMP.

In some embodiments, the cAMP-elevating agent is forskolin, cholera toxin, dibutyryl cAMP, isobutylmethylxanthine, theophylline, isoproteronol, or PGE2. In some embodiments, the cAMP-elevating agent is forskolin.

cAMP-elevating agents are available commercially, e.g. from Sigma (St. Louis, Mo.), and may be used at concentrations approximating those described in Green (Proc. Natl. Acad. Sci. USA 15:801-811 (1978)), which is incorporated herein by reference in its entirety.

Any of the above-listed compounds useful in the subject methods may be modified to increase the bioavailability, activity, or other pharmacologically relevant property of the compound. For example, forskolin has a formula as shown below:

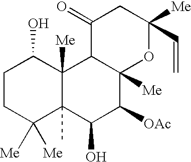

Modifications of forskolin which have been found to increase the hydrophilic character of forskolin without severely attenuating the desired biological activity include acylation of the hydroxyls at C6 and/or C7 (after removal of the acetyl group) with hydrophilic acyl groups. In compounds wherein C6 is acylated with a hydrophilic acyl group, C7 may optionally be deacetylated. Suitable hydrophilic acyl groups include groups having the structure—(CO)(CH₂)ₙ X, wherein X is OH or NR₂; R is hydrogen, a C₁-C₄ alkyl group, or two Rs taken together form a ring comprising 3-8 atoms, preferably 5-7 atoms, which may include heteroatoms (e.g., piperazine or morpholine rings); and n is an integer from 1-6, preferably from 1-4, even more preferably from 1-2. Other suitable hydrophilic acyl groups include hydrophilic amino acids or derivatives thereof, such as aspartic acid, glutamic acid, asparagine, glutamine, serine, threonine, tyrosine, etc., including amino acids having a heterocyclic side chain. Forskolin, or other compounds listed above, modified by other possible hydrophilic acyl side chains known to those of skill in the art may be readily synthesized and tested for activity in the present method.

Similarly, variants or derivatives of any of the above-listed compounds may be effective as cAMP agonists in the subject method. Those skilled in the art will readily be able to synthesize and test such derivatives for suitable activity.

In certain embodiments, the subject cAMP agonists can be chosen on the basis of their selectivity for cAMP activation.

In certain embodiments, it may be advantageous to administer two or more of the above cAMP-elevating agents, preferably of different types. For example, use of an adenylate cyclase agonist in conjunction with a cAMP phosphodiesterase antagonist may have an advantageous or synergistic effect.

Compositions and methods of the invention utilize a given cAMP-elevating agent at a suitable concentration. In some embodiments of cell culture medium and cell culture methods, the cAMP-elevating agent is forskolin present at a concentration of about 0.02-200 ug/ml, or about 0.2-20 ug/ml, or about 0.4-10 ug/ml, or about 0.8-5 ug/ml, or about 1.5-2.0 ug/ml, or about 1.0, 1.1, 1.2, 1.3, 1.4, 1.5, 1.6, 1.7, 1.8, 1.9, 2.0, 2.1, 2.2, 2.3, 2.4, or 2.5 ug/ml. In some embodiments of cell culture medium and cell culture methods, the cAMP-elevating agent is cholera toxin present at a concentration of about 0.001-10 ug/ml, or about 0.01-1 ug/ml, or about 0.05-0.5 ug/ml, or about 0.08-0.1 ug/ml, or about 0.02, 0.03, 0.04, 0.05, 0.06, 0.07, 0.08, 0.09, 0.10, 0.11, 0.12, 0.13, or 0.14 ug/ml. In some embodiments of cell culture medium and cell culture methods, the cAMP-elevating agent is dibutyryl cAMP present at a concentration of about 1.5-15,000 ug/ml, or about 15-1500 ug/ml, or about 30-750 ug/ml, or about 60-300 ug/ml, or about 100-200 ug/ml or about 100, 110, 120, 130, 140, 150, 160, 170, 180, 190, or 200 ug/ml. In some embodiments of cell culture medium and cell culture methods, the cAMP-elevating agent is isobutylmethylxanthine (IBMX) present at a concentration of about 0.07-700 ug/ml, or about 0.7-70 ug/ml, or about 1.5-35 ug/ml, or about 3.5-15 ug/ml, or about 5-10 ug/ml, or about 1, 2, 3, 4, 5, 6, 7, 8, 9, 10, 11, 12, 13, 14, or 15 ug/ml. In some embodiments of cell culture medium, the cAMP-elevating agent is theophylline present at a concentration of about 0.07-700 ug/ml, or about 0.7-70 ug/ml, or about 1.5-35 ug/ml, or about 3.5-15 ug/ml, or about 5-10 ug/ml, or about 1, 2, 3, 4, 5, 6, 7, 8, 9, 10, 11, 12, 13, 14, or 15 ug/ml. In some embodiments of cell culture medium, the cAMP-elevating agent is isoproterenol present at a concentration of about 0.003-30 ug/ml, or about 0.03-3 ug/ml, or about 0.06-1.5 ug/ml, or about 0.1-1 ug/ml, or about 0.05, 0.1, 0.15, 0.2, 0.25, 0.3, 0.35, 0.4, 0.45, 0.5, 0.55, or 0.6 ug/ml. In some embodiments of cell culture medium, the cAMP-elevating agent is PGE₂ present at a concentration of about 0.001-10 ug/ml, or about 0.01-1 ug/ml, or about 0.05-10 ug/ml, or about 0.001-10 ug/ml, or about 0.01-1 ug/ml, or about 0.05, 0.1, 0.15, 0.2, 0.25, 0.3, 0.35, 0.4, 0.45, 0.5, 0.55, or 0.6 ug/ml.

After about the first 24-48 hours of cell culture, fibroblasts float to the top of the culture medium and can be removed by washing the keratinocytes (which adhere to the culture apparatus, e.g., plate). The fibroblasts may be discarded or they may be cultured separately. After about two days, the culture is generally >99% keratinocytes, because of the apoptosis and removal of fibroblasts. In contrast, when using conventional methods of keratinocyte culture, even when just the epidermis is taken, significant fibroblast contamination can be seen in the culture. See, e.g., FIGS. 1 and 2. The degree of contamination can be quantitated by observing gene expression for genes expressed mainly by fibroblasts, and/or by examining the ratio of a gene expressed at high levels in fibroblasts gene to a gene expressed at low levels by keratinocytes. The latter method is useful when keratinocyte concentrations in the culture have become very high. In some embodiments, the methods of the invention comprise the production of cultures of keratinocytes that comprise at least about 60, 70, 80, 90, 95, 98, 99, 99.5, 99.9, or 99.99% keratinocytes. In some embodiments, the methods of the invention comprise the production of cultures of keratinocytes that comprise between about 90 and 99.99% keratinocytes, or between about 95 and 99.99% keratinocytes, or between about 98 and 99.99% keratinocytes, or between about 99 and 99.99% keratinocytes. In some embodiments, the methods of the invention comprise the production of cultures of keratinocytes that comprise between about 90 and 100% keratinocytes, or between about 95 and 100% keratinocytes, or between about 98 and 100% keratinocytes, or between about 99 and 100% keratinocytes.

Cells are passed when about 60-70% confluent. It is useful for tissue culture not to let the cells grow to or near confluence, as cells at or near 100% confluence produce a factor that encourages fibroblast growth. In some embodiments, cells are passaged when they are between about 40% to 90% confluent, or between about 50% to 80% confluent, or between about 60% to 70% confluent. In some embodiments, cells are passaged when they are about 50%, or 55%, or 60%, or 65%, or 70%, or 75%, or 80% confluent. In some cases, it may be wished to grow cells to greater degrees of confluence than 60-70% in order to obtain medium for certain therapeutic or other uses.

Cells can be passaged for at least two years. This is considerably different from normal keratinocyte culture, where, because of fibroblast proliferation, the cells must be used earlier. Some embodiments of the invention provide cultures that are at least about 90, 95, 98, 99, 99.5, 99.9, or 99.99% keratinocytes, where the culture persists for at least about 1 month, 2 months, 3 months, 6 months, or about 9 months, or about 12 months, or about 15 months, or about 18 months, or about two years, or about two and one-half years, or about three years, or about four years, or more than about four years.

IV. Compositions

In one aspect the invention provides compositions that include conditioned medium from keratinocytes, and/or one or more proteins from conditioned medium from keratinocytes. The compositions can also contain a cAMP-elevating agent. These compositions include culture media for tissue (e.g., keratinocyte) culture, and compositions for therapeutic use (e.g., wound healing) and/or cosmetic use. In some embodiments, the conditioned medium is from cultures of fetal keratinocytes, e.g., fetal keratinocytes cultured in the presence of keratinocyte-conditioned medium.

A. Conditioned Medium

In some embodiments, the invention provides conditioned medium from cultured keratinocytes, typically keratinocytes that have themselves been cultured in the presence of conditioned medium from keratinocytes. As used herein, “conditioned medium” includes both medium that is used as is (i.e., as removed from a cell culture) as well as medium that has been treated to concentrate the medium, remove unwanted proteins or other substances, etc. In one embodiment the invention provides conditioned medium from fetal keratinocytes that have been cultured in the presence of keratinocyte-conditioned medium. The conditioned medium in some embodiments is produced in cultures where the pre-conditioned medium (e.g., the fresh medium added at intervals during keratinocyte culture) is serum-free or animal-product-free; in some embodiments the pre-conditioned medium is a defined medium. In some embodiments the conditioned medium is produced by a keratinocyte culture from adult keratinocytes, or neonatal keratinocytes, or fetal keratinocytes.

For therapeutic use, conditioned medium from fetal keratinocytes may have advantages, as discussed below. As is known in the art, and as can be readily ascertained by the skilled practitioner for a given culture type and conditions, the profile of the conditioned medium (e.g., type and concentration of compounds and substances in the medium) changes based on the type of keratinocyte in culture, the length of time cells have been cultured in the medium, degree of confluence of cells in the medium, degree of keratinocyte purity, and other factors apparent to those of skill in the art. Thus, the medium may be removed from culture at any time, degree of confluence, and/or degree of keratinocyte purity, or any other factor, that is appropriate, based on the subsequent intended use of the medium. Alternatively, the medium may be removed from the culture when a given factor(s) or substance(s) reaches a particular concentration. In some embodiments, the conditioned medium is produced in a keratinocyte culture that is at least about 60%, 70%, 80%, 90%, 95%, 98%, 99%, 99.5%, 99.9%, or 99.99%, or about 100% keratinocytes. In some embodiments, the medium is produced in a keratinocyte culture that is between about 10% to 90% confluent, or between about 20% to 80% confluent, or between about 30% to 80% confluent, or between about 40% to 80% confluent, or between about 50% to 70% confluent, or between about 60% to 70% confluent. In some embodiments, the medium is produced in a culture, or removed from culture, when the cells are about 50%, or 55%, or 60%, or 65%, or 70%, or 75%, or 80% confluent. Thus, in some embodiments, medium is produced in a culture that is about 90% to about 100% pure keratinocytes (e.g., fetal keratinocytes) and is removed from the culture when cells are about 50% to 70% confluent; in some embodiments medium is produced in a culture that is about 95% to about 100% pure keratinocytes (e.g., fetal keratinocytes) and is removed from the culture when cells are about 55% to 70% confluent; in some embodiments, medium is produced in a culture that is about 98% to about 100% pure keratinocytes (e.g., fetal keratinocytes) and is removed from the culture when cells are about 60% to 70% confluent.

In some embodiments, the medium is removed from culture between about 1 to about 100 hours after fresh medium has been added to the culture, or between about 10 to about 75 hours after fresh medium has been added to the culture, or between about 10 to about 50 hours after fresh medium has been added to the culture, or between about 15 to about 50 hours after fresh medium has been added to the culture, or between about 20 to about 40 hours after fresh medium has been added to the culture. In some embodiments the medium is removed after about 1, 2, 3, 4, 5, 6, or 7 days from the time of addition of fresh medium to the culture. In some embodiments, conditioned media from different cultures, or from the same culture gathered at different times, are combined. Any suitable combination of cell types, duration of culture, confluence, purity of keratinocytes, and the like, may be used in embodiments of the invention.

The keratinocytes are cultured by methods of the invention as described herein. In some embodiments, the keratinocytes are cultured in an environment that enables aseptic processing and handling. In some embodiments, the medium is conditioned in a manner allowing for large scale growth (yielding large amounts of conditioned medium) using techniques known in the art, e.g., an apparatus for aseptic large scale culturing like that described in U.S. Pat. Nos. 5,763,267 5,843,766 (also incorporated herein in its entirety) which describes an apparatus for aseptic growth of three-dimensional tissue cultures. Using an aseptic closed system such as described in the '267 patent, preconditioned culture media is transported from a fluid reservoir to an inlet manifold and evenly distributed to the cultures in a continuous flow system. When appropriate, (i.e., once the media is conditioned so that the extracellular factors, e.g. proteins have reached desirable levels in the media) it is pumped out of the system and processed for use.

Conditioned medium removed from the cell culture apparatus may be used as is or may be further processed. Such processing may include, but is not limited to, dilution, and concentration by a water flux filtration device or by defiltration using the methods described in Cell & Tissue Culture: Laboratory Procedures, supra. Additionally, the conditioned medium may be further processed for product isolation and purification to remove unwanted substances, e.g., proteases. The methods used for product isolation and purification so that optimal biological activity is maintained will be readily apparent to one of ordinary skill in the art. For example, it may be desirous to purify a growth factor, regulatory factor, peptide hormone, cytokine, etc. Methods for product isolation and purification include, but are not limited to, gel chromatography (using matrices such as Sephadex) ion exchange, metal chelate affinity chromatography with an insoluble matrix such as cross-linked agarose, HPLC purification and hydrophobic interaction chromatography of the conditioned media. Such techniques are described in greater detail in Cell & Tissue Culture; Laboratory Procedures, and in other standard textbooks. Depending upon the desired application of the conditioned medium, and/or products derived thereof, appropriate measures can be taken to maintain sterility. Alternatively, sterilization may be necessary and can be accomplished by methods known to one of ordinary skill in the art, such as, for example, heat and/or filter sterilization taking care to preserve the desired biological activity. Conditioned cell media of the invention may be concentrated and/or lyophilized.

B. Culture Media

In one aspect the invention provides culture medium for culturing keratinocytes, where the culture medium itself contains conditioned medium from the culture of keratinocytes, e.g., fetal keratinocytes. The conditioned medium may be as described above. It may be used as it is harvested, or, more typically, processed for use in culture medium. The processing may include any or all of: removal of cellular debris or other particulate matter, as well as proteases, lactic acid and other components possibly detrimental to cells; concentration; dilution; addition of other substances, and the like. In some embodiments, the conditioned culture medium is purified to become enriched in one or more proteins or other components, then added to non-conditioned medium. In some embodiments, the invention provides a culture medium produced by mixing fresh (non-conditioned) culture medium with a protein produced by the process of culturing keratinocytes, e.g., fetal keratinocytes. The culture medium of the invention containing keratinocyte-conditioned medium can contain at least about 10, 20, 30, 40, 50, 60, 70, 80, 90, or 95% keratinocyte-conditioned medium. In some embodiments, culture medium of the invention containing keratinocyte-conditioned medium contains between about 10% and about 95% keratinocyte-conditioned medium, or between about 20% and about 90% keratinocyte-conditioned medium, or between about 30% and about 80% keratinocyte-conditioned medium, or between about 40% and about 75% keratinocyte-conditioned medium, or between about 45% and about 70% keratinocyte-conditioned medium, or between about 50% and about 70% keratinocyte-conditioned medium or between about 55% and about 70% keratinocyte-conditioned medium or between about 50% and about 65% keratinocyte-conditioned medium or between about 55% and about 65% keratinocyte-conditioned medium or between about 60% and about 70% keratinocyte-conditioned medium. In some embodiments, culture medium of the invention containing keratinocyte-conditioned medium contains about 10, 20, 30, 40, 45, 50, 52, 55, 56, 57, 58, 59, 60, 61, 62, 63, 64, 65, 67, 70, 75, 80, 85, 90, or 95% keratinocyte-conditioned medium.

The keratinocyte-conditioned medium may be mixed with any appropriate medium or composition to produce a culture medium of the invention. Thus, the keratinocyte-conditioned medium may be mixed with pre-made keratinocyte cell culture medium, such as those commercially available (e.g., with serum, serum-free, animal-product free, or defined). Suitable keratinocyte cell culture media for mixture with the conditioned medium include those recited herein for use in keratinocyte culture. It will be appreciated that a keratinocyte culture medium can also be prepared “from scratch” using art-accepted techniques, and admixed with conditioned medium.

Additional ingredients may be added to the culture medium as well. In one embodiment, the culture medium contains an exogenously-added cAMP-elevating agent. Such agents and appropriate concentrations are described above. Other ingredients include, without limitation, amino-acids (both D and/or L-amino acids) such as glutamine, alanine, arginine, asparagine, cystine, glutamic acid, glycine, histidine, isoleucine, leucine, lysine, methionine, phenylalanine, proline, serine, threonine, tryptophan, tyrosine, and valine and their derivatives; acid soluble subgroups such as thiamine, ascorbic acid, ferric compounds, ferrous compounds, purines, glutathione and monobasic sodium phosphates. Additional ingredients include sugars, deoxyribose, ribose, nucleosides, water soluble vitamins, riboflavin, salts, trace metals, lipids, acetate salts, phosphate salts, HEPES, phenol red, pyruvate salts and buffers. Other ingredients often used in media formulations include fat soluble vitamins (including A, D, E and K) steroids and their derivatives, cholesterol, fatty acids and lipids Tween 80, 2-mercaptoethanol pyramidines as well as a variety of supplements including serum (fetal, horse, calf, etc.), proteins (Insulin, transferrin, growth factors, hormones, etc.) antibiotics (gentamicin, penicillin, streptomycin, amphotericin B, etc.) whole egg ultra filtrate, and attachment factors (fibronectins, vitronectins, collagens, laminins, tenascins, etc.). The concentrations of the ingredients are well known to one of ordinary skill in the art. See, for example, Methods For Preparation Of Media, Supplements and Substrate for Serum-free Animal Cell Cultures, supra.

Culture media of the invention may also include BPE. If BPE is added, it may be used at any concentration appropriate to the culture of keratinocytes. Exemplary concentrations are about 1 to about 100 ug/ml, or about 5 to about 75 ug/ml, or about 10 to about 50 ug/ml, or about 10, 15, 20, 25, 30, 35, 40, 45, or 50 ug/ml. In some embodiments, BPE is used at 30 ug/ml.

Although culture media of the invention are advantageously prepared without serum (serum-free), in some embodiments it may be desirous to use serum in the culture medium.

Various embodiments of the culture media of the invention thus include: A culture medium that contains conditioned medium from keratinocyte culture, e.g., fetal keratinocyte culture, at about 55% and about 65% keratinocyte-conditioned medium; an exogenously-added cAMP-elevating agent, e.g., forskolin, at about 1.5-2.0 ug/ml; and unconditioned medium (e.g., medium that has not been used for cell culture), where the unconditioned medium is serum-containing, serum-free, animal product-free, or defined. Culture media containing serum, or serum-free, may further contain BPE at about 10 to about 50 ug/ml. In one embodiment, culture medium of the invention contains about 40% conditioned medium from the culture of fetal keratinocytes, where the fetal keratinocytes were allowed to grow to about 70% confluence; about 2 ug/ml forskolin; and 60% unconditioned medium, where the medium is Serum Free Medium or Defined medium (GIBCO or EPILIFE (CASCADE).

In some embodiments, culture medium of the invention is concentrated, and is used by the consumer in diluted form. Methods of concentration and appropriate diluents are well-known in the art.

In some embodiments, the keratinocyte culture medium is packaged for transport, storage and/or use by a consumer. Such packaging of tissue culture medium for transport, storage, and/or use is well-known in the art. Packaged medium may include further components for the dispensing and storage of the medium, and may also include separately packaged diluent for dilution of concentrated medium, optional additional ingredients for inclusion by the user if desired, instructions for use, and the like.

C. Therapeutic and Cosmetic Compositions

The invention also provides therapeutic and cosmetic compositions and methods. It is well-known that wounded skin from neonates heals with little scarring, and fetal skin heals with little or no scarring. Conditioned medium from keratinocytes, e.g., adult, neonatal or, especially, fetal keratinocytes, contains factors that promote scarless healing. Without being bound by theory, it is thought that the conditioned medium contains one or more factors that inhibit fibroblast growth, and that the presence of one or more of these factors in the wound environment promotes healing by, at least in part, inhibiting fibroblast formation on a wound, leading to less scarring (from fibroblasts) and more smooth skin. Such factors, and other components of keratinocyte conditioned medium, may also contribute to cosmetic uses for, e.g., treatment of wrinkles, skin aging, and the like.

Conditioned medium may be used as is, or, more typically treated to enrich the medium in one or more factors that are therapeutic and/or useful for cosmetic purposes, remove undesirable or unnecessary factors (e.g., by filtration, dialysis, centrifugation, chromatography, etc.), concentrate the medium, sterilize, add additional ingredients, and/or treat for storage and transport. As used herein, “conditioned medium” includes both medium that is used as is (as removed from culture medium) as well as medium that has been treated as discussed herein. In some embodiments, one or more cAMP-elevating agents may be added to the conditioned medium.

In some embodiments, the conditioned medium is from keratinocytes, e.g., adult, neonatal, or fetal keratinocytes, that have been cultured entirely in serum-free medium. In some embodiments, the conditioned medium is from keratinocytes, e.g., adult, neonatal, or fetal keratinocytes, that have been cultured entirely in animal product-free medium.

In some embodiments of the invention, the conditioned cell medium compositions of the invention are used for pharmaceutical applications. In some embodiments, the conditioned cell medium compositions are used in compositions and methods to promote wound and burn healing. Some embodiments of the invention provide a composition for promoting wound healing that includes a protein that is produced by growing keratinocytes in culture. In some embodiments the keratinocytes are fetal keratinocytes.

Wound healing is a complex process that involves several stages and is capable of sealing breaches to the integument in a controlled manner to form functionally competent tissue. The process begins with hemostasis followed by an inflammatory phase involving neutrophils and macrophages. The process continues with the development of granulation tissue and re-epithelialization to close the wound. Subsequently, scar tissue forms and is remodeled over the succeeding months to an approximation of the original anatomical structure. Ideally, scar tissue is minimal so that healthy tissue, functionally competent tissue which histologically and physiologically resembles the original normal tissue, may form. Each stage of the healing process is controlled by cellular interactions through regulatory proteins such as cytokines, growth factors, and inflammatory mediators as well as cell contact mechanisms. For example, inflammatory mediators such as IL-6, IL-8, and G-CSF induce lymphocyte differentiation and acute phase proteins, as well as neutrophil infiltration, maturation and activation, processes that are important in the inflammatory stages of wound healing. Other examples of regulatory proteins involved in the wound healing process are VEGF that induces angiogenesis during inflammation and granulation tissue formation, the BMP's which induce bone formation, KGF that activates keratinocytes and TGFβ1 that induces deposition of extracellular matrix.

In chronic wounds, the healing process is interrupted at a point subsequent to hemostasis and prior to re-epithelialization, and is apparently unable to restart. Most of the inflammation seen in the wound bed is related to infection, but the inflammation gives rise to an environment rich in proteases that degrade regulatory proteins and thus interfere with the wound healing process. Similarly, in some medical conditions, such as diabetes, some of the regulatory proteins needed for wound healing are in short supply. For example, it has been found in a mouse model of non-insulin-dependent diabetes (e.g., the db/db mouse) that secretion of VEGF and PDGF and expression of the PDGF receptor are all depressed in wounds compared to the levels in wounds of normal mice.

Without being bound by theory, it is thought that the conditioned media of the present invention is useful in wound healing because it one or more of the regulatory proteins thought to be important in wound healing and which have been shown to be depleted in vivo models of wound healing. Similarly, the conditioned media provided by the present invention is also useful in the treatment of other types of tissue damage, e.g., traumatic or congenital, wherein the repair and/or regeneration of tissue defects or damage is desired since many of these growth factors are found in keratinocyte conditioned cell media, including, for example, fibroblast growth factors (FGFs), platelet derived growth factors (PDGFs), epidermal growth factors (EGFs), bone morphogenetic proteins (BMPs) and transforming growth factors (TGFs); as well as those which modulate vascularization, such as vascular endothelial growth factor (VEGF), keratinocyte growth factor (KGF), and basic FGF; angiogenesis factors, and antiangiogenesis factors. Stress proteins, such as GR 78 and MSP90 induce growth factors such as TGF-β. TGF-β, including TGF β-1, TGF β-2, TGF β-3, TGF β-4 and TGF β-5, regulate growth and differentiation and accelerate wound healing. Mitogens, such as PDGF increase the rate of cellularity and granulation in tissue formation.

Because the conditioned medium contains such an array of wound healing factors, the conditioned medium of the invention is advantageously used in the treatment of wound and burn healing including skin wounds, broken bones, gastric ulcers, pancreas, liver, kidney, spleen, blood vessel injuries and other internal wounds. Further, the conditioned medium may be combined with other medicinal ingredients such as antibiotics and analgesics. Embodiments include formulations of the conditioned media with a salve or ointment for topical applications. Keratinocytes used to produce conditioned medium for use in therapeutic compositions for use in humans are preferably human to minimize immunogenicity problems.

Examples wound healing compositions and methods of the invention include, but are not limited to, applying the conditioned cell medium, in some embodiments with exogenous cAMP-elevating agent, to the gauze of a bandage (adhesive or non-adhesive) and using conditioned medium of the invention in topical applications to promote and/or accelerate wound healing. Again, the conditioned medium may be processed to concentrate or reduce one or more components to enhance wound healing. The compositions may be lyophilized/freeze-dried and added as a wound filler or added to existing wound filling compositions to accelerate wound healing. Alternatively, the medium may be added to a hydrogel composition and used as a film for topical wound treatments and anti-adhesion applications.

The conditioned media may be used in any state, i.e., liquid or solid, frozen lyophilized or dried into a powder, as a film for topical wound treatments and anti-adhesion applications, as an injectable, see PCT WO 96/39101, incorporated herein by reference it its entirety.

Alternatively, the conditioned cell medium of the present invention may be formulated with polymerizable or cross-linking hydrogels as described, e.g., in U.S. Pat. Nos. 5,709,854; 5,516,532; 5,654,381; and PCT Publications No. WO 94125080 and WO 98/52543, each of which is incorporated herein by reference in its entirety. Examples of materials which can be used to form a hydrogel include modified alginates, polysaccharides, covalently cross-linkable hydrogel precursors (e.g., acrylates, diacrylates, oligoacrylates, dimethacrylates, oligomethacrylates, and other biologically acceptable photopolymerizable groups), and the like. Polymers useful in wound healing compositions comprising conditioned medium are further described in U.S. Pat. No. 6,372,494, which is hereby incorporated by reference herein in its entirety.

In another embodiment, the conditioned media, or particular factors elaborated into the media, are used to provide a substance to coat sutures. Similarly, the conditioned cell media of the invention or particular factors elaborated into the media may be used to coat conventional implantation devices, including vascular prosthesis, in surgical approaches to correct defects in the body. The implants can be made of, e.g., biocompatible, inert materials, that replace or substitute for the defective function and can be made of either non-biodegradable materials or biodegradable materials. By coating implantation devices with the medium or factors derived therefrom, the implant invites proper cellular attachments resulting in superior tissue at the implantation site. Thus, without being bound by theory, sutures, bandages, and implants coated with conditioned cell media, or one or more factors (e.g., proteins) derived form the media, enhance the recruitment of cells, such as leukocytes and fibroblasts into the injured area and induce cell proliferation and differentiation resulting in improved wound healing.

In another embodiment, the medium or factor(s) derived therefrom may be formulated with a pharmaceutically acceptable carrier as a vehicle for internal administration. Also, the medium may be further processed to concentrate or reduce one or more factor or component contained within the medium, for example, enrichment of a growth factor using immunoaffinity chromatography or, conversely, removal of a less desirable component, for any given application as described herein.

The conditioned cell medium or factor(s) derived therefrom may also be added to devices used in periodontal surgery in order to promote uniform tissue repair. In another embodiment, the compositions may be lyophilized/freeze-dried and added as a wound filler (e.g., fill holes left from hair plugs for implantation) or added to existing wound filling compositions to accelerate wound healing.

The invention also provides methods of promoting wound or burn healing comprising contacting the wound or burn with one or more factors derived from or contained in conditioned medium, where the conditioned medium is obtained from the culture of keratinocytes. In some embodiments, the conditioned medium is from keratinocytes that have themselves been cultured in the presence of conditioned medium. In some embodiments, the factor is a protein. In some embodiments, the invention provides methods of promoting the healing of burns that include culturing keratinocytes for use in the treatment of a burn by a method that includes exposing the keratinocytes to keratinocyte-conditioned medium (e.g., from fetal keratinocytes), growing the keratinocytes, and applying the keratinocytes to the site of the burn.

In some embodiments, conditioned media of the invention may be used for cosmetic purposes. For cosmetic applications, the compositions may be added in any form and may be used in a hydrogel, injectable, cream, ointment, and may even be added to eye shadow, pancake makeup, compacts or other cosmetics to fortify the skin topically. Further, formulations in the form of injectables or hydrogels may be used to eliminate wrinkles, frown lines, scanning and to repair other skin conditions. The conditioned medium may be used instead of, or in addition to, e.g., using silicone or other products to do so.

V. Kits

In another aspect, the invention provides kits. The kits can include any of the compositions of the invention, together with, e.g. packaging material, additional compositions, instructions, promotional or sales material, and the like.

Compositions of the invention that may be included in kits of the invention include keratinocyte conditioned medium, keratinocyte culture medium, one or more cAMP-elevating agents, therapeutic or pharmaceutical compositions such as those used for the treatment of wounds and/or burns, and cosmetic compositions. Additional compositions that can be included in the kits of the invention include cells (e.g., for cell culture), which may be keratinocytes from any source as described herein. Also useful are buffers and diluents (e.g., for diluting or modifying keratinocyte conditioned medium and/or keratinocyte culture medium and/or for reconstituting lyophilized compositions). Further useful components of kits include apparatus or substances helpful in utilizing compositions of the invention. These include without limitation culture dishes, pipettes, other cell culture equipment, applicators, syringes, bandages, tape, scissors, gauze, brushes, equipment and materials for preparation and/or clean-up and proper disposal from use of compositions of the invention for, e.g., wound, burn, or cosmetic application, materials for dispensing and storing compositions of the invention, and other materials for use in conjunction or in addition to the compositions of the invention. Kits may also include additional therapeutic compositions (e.g., for the treatment of wounds, burns, and the like) such as antibiotics, palliative salves, ointments, creams and the like, anesthetics, analgesics, and other compositions useful in the treatment of wounds and burns. Kits that comprise cosmetic compositions may further include materials for application and clean-up as appropriate to the composition.

The kits herein can include at least one composition of the invention and a set of written instructions. Instructions can include information concerning the compositions of the kit, including information on adverse effects, suspected mechanisms of action, dosages, means of application, means of clean-up and disposal, information required by regulatory agencies, indications for use, counterindications, and the like. The instructions may be in any form, whether written, electronic, or other; instructions may include a designation for a website and the contents of the website.

VI. Business Methods

The invention also provides business methods.

In some embodiments, a therapeutic comprising a composition of the invention, e.g., in kit form, may be marketed to a consumer, who may be a medical professional, pharmacy, or end-user, and payment may be received for the therapeutic. In some embodiments, keratinocyte-conditioned medium or a composition containing keratinocyte-conditioned medium, e.g., keratinocyte culture medium, e.g., in kit form, may be marketed to a consumer, and payment received for the medium. In some embodiments, a cosmetic composition, e.g., in kit form, may be marketed to a consumer, and payment received for the cosmetic composition. In some embodiments, business methods of the invention include methods of providing customized cell culture media to a customer, where the needs of the customer for media are ascertained, the media is produced, and the media is transported to the customer. In some embodiments, the customized cell culture medium contains conditioned medium from the culture of keratinocytes.

### EXAMPLES

### Example 1

### Preparation of Keratinocytes and Culture of Keratinocytes

Skin was cleaned, removed from the animal, and placed in DMEM medium. In various procedures, the skin was from fetal, newborn, and adult animals. The type of animal include mouse (fetal and adult), rat (fetal and adult) and human (neonatal foreskin). The skin was chopped, then placed in digestive medium. The digestive medium can contain any of the common digestive enzymes, e.g. collagenase. In these procedures, the skin was digested in 0.3% Dispase & trypsin 0.2% (1/1 dilution) at 37 C for 1-2 hours. This is much shorter than the normal digestion, which is overnight. The digestive mixture was mixed every 15-20 minutes. After 1-2 hours, the cells were dissociated with a 10 ml glass pipette, then passed through a 75 μm cell strainer. Cells were washed in DMEM medium and centrifuged at 800 rpm for 5-10 minutes at least three times.

The cells were then plated in GIBCO keratinocyte Serum Free Medium (SFM) with forskolin at 2 ug/ml. In some procedures, this medium was used with bovine pituitary extract and in some procedures it was not. The use of BPE is not critical.

The medium was replaced daily for the next 5 days; when the medium was replaced, it was replaced with 50% conditioned medium (from the cell culture) and 50% fresh GIBCO SFM with forskolin. After the first 24-48 hrs fibroblasts, which float to the top, were removed. After about two days, the cultures were >99% keratinocytes, because of the apoptosis and removal of fibroblasts. Cells were passed when 60-70% confluent. The cells can be passaged for at least two years.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

All publications and patent applications mentioned in this specification are herein incorporated by reference to the same extent as if each individual publication or patent application was specifically and individually indicated to be incorporated by reference.

What is claimed is:

1. A method of culturing keratinocytes comprising contacting growing keratinocytes with a culture medium comprising conditioned medium, wherein said conditioned medium has previously been used to culture fetal keratinocytes.

2. The method of claim 1 further comprising contacting the growing keratinocytes with a cyclic adenosine monophosphate (cAMP)-elevating agent.

3. The method of claim 2 wherein the cAMP-elevating agent is selected from the group consisting of forskolin, cholera toxin, dibutyryl cAMP, isobutylmethylxanthine, theophylline, isoproterenol, and PGE2.

4. The method of claim 3 wherein the cAMP-elevating agent is forskolin.

5. The method of claim 4 wherein the forskolin is present at a concentration of about 0.8 ug/ml to about 5 ug/ml.

6. The method of claim 1 wherein the growing keratinocytes are fetal keratinocytes.

7. The method of claim 6 wherein the growing fetal keratinocytes and the fetal keratinocytes used to produce the conditioned medium are genetically identical.

8. The method of claim 1 wherein the culture medium comprises about 30-50% conditioned medium.

9. The method of claim 1 wherein the culture medium is produced by the steps of:
(a) culturing fetal keratinocytes in a keratinocyte culture medium for at least about eight hours to produce a conditioned medium; and
(b) mixing the conditioned medium with fresh keratinocyte culture medium so that the conditioned medium comprises about 30-50% by volume of the mixture.

10. The method of claim 9 further comprising adding a cAMP-elevating agent to the culture medium.

11. The method of claim 10 wherein step (b) is repeated about every day.

12. The method of claim 11, wherein step (b) is repeated until a cell culture is produced wherein the cells of the cell culture comprise greater than about 99% keratinocytes.

13. The method of claim 11, wherein step (b) is repeated until a cell culture is produced wherein the cells of the cell culture comprise greater than about 99.9% keratinocytes.

14. The method of claim 1 further comprising passing the keratinocytes when they are about 60-70% confluent.

15. The method of claim 1 wherein the fetal keratinocytes are grown in serum-free medium.

16. A method of culturing keratinocytes comprising contacting growing keratinocytes with a culture medium comprising conditioned medium, wherein said conditioned medium has previously been used to culture keratinocytes under animal product-free conditions at all times.

17. The method of claim 16 further comprising contacting the growing keratinocytes with a cAMP-elevating agent.

18. A composition for culturing keratinocytes comprising keratinocyte culture medium and conditioned medium in which fetal keratinocytes have been cultured.

19. The composition of claim 18 further comprising a cAMP-elevating agent.

20. The composition of claim 19 wherein the cAMP-elevating agent is selected from the group consisting of forskolin, cholera toxin, dibutyryl cAMP, isobutylmethylxanthine, theophylline, isoproterenol, and PGE2.

21. The composition of claim 20 wherein the cAMP-elevating agent is forskolin.

22. The composition of claim 21 wherein the forskolin is present at a concentration of about 0.8 ug/ml to about 5 ug/ml.

23. The composition of claim 18 wherein the conditioned medium comprises about 30-50% of the composition by volume.

24. A culture medium produced by mixing fresh culture medium with a protein produced by the process of culturing fetal keratinocytes.

25. The composition of claim 24 further comprising a cAMP-elevating agent.

26. A cell culture medium comprising conditioned medium that has previously been used to culture keratinocytes under conditions wherein said cultured keratinocytes are cultured at all times under animal product-free conditions.

27. The composition of claim 26 further comprising a cAMP-elevating agent.

28. A keratinocyte culture that persists for more than about one year, wherein the cells of the culture comprise at least about 99% keratinocytes.

29. A composition for promoting wound healing comprising a protein that is produced by growing fetal keratinocytes in culture.

30. A composition for promoting wound healing comprising a protein that is produced by growing keratinocytes in culture, wherein the keratinocytes are grown by a method comprising contacting growing keratinocytes with a culture medium comprising conditioned medium, wherein said conditioned medium has previously been used to culture keratinocytes under conditions wherein said cultured keratinocytes are cultured at all times under animal product-free conditions.

31. A method of modulating wound healing comprising contacting a wound with a protein from a conditioned medium produced by fetal keratinocyte culture.

32. The method of claim 31 further comprising contacting the wound with a cAMP-elevating agent.

33. The method of claim 32 wherein the cAMP-elevating agent is forskolin.
